(19) United States
(12) Patent Application Publication
(10) Pub. No.: US 2025/0103630 A1
(43) Pub. Date: Mar. 27, 2025

(54) SUPPORT APPARATUS, SUPPORT METHOD, AND SUPPORT PROGRAM

(71) Applicant: FUJIFILM Corporation, Tokyo (JP)

(72) Inventors: Shotaro MISAWA, Tokyo (JP); Shintaro OYAMA, Nagoya (JP); Taiki FURUKAWA, Nagoya (JP); Hirokazu YARIMIZU, Tokyo (JP); Kohei ONODA, Tokyo (JP); Nobuya KITAMURA, Tokyo (JP)

(73) Assignee: FUJIFILM Corporation, Tokyo (JP)

(21) Appl. No.: 18/892,555

(22) Filed: Sep. 23, 2024

(30) Foreign Application Priority Data
Sep. 27, 2023 (JP) 2023-166471

(51) Int. Cl. G06F 16/33; G16H 10/60

(52) U.S. Cl. CPC G06F 16/3344; G16H 10/60

(57) ABSTRACT

A document creation support apparatus acquires document information associated with an input target person, derives the number of pieces of examination information included in the document information for each examination item, and derives a degree of importance of the examination item for the target person based on the derived number of pieces of the examination information.

### CROSS-REFERENCE TO RELATED APPLICATIONS

This application claims priority from Japanese Patent Application No. 2023-166471, filed on Sep. 27, 2023, the entire disclosure of which is incorporated herein by reference.

### BACKGROUND

### 1. Technical Field

The present disclosure relates to a support apparatus, a support method, and a support program.

### 2. Description of the Related Art

JP2012-141797A discloses a technology for automatically searching for a highly related medical report from a past medical report using, as a keyword, a word or a word sequence that is weighted by a frequency of occurrence and a degree of importance related to a medical case for automatically searching for a related medical case report from a sentence of a past medical report.

### SUMMARY

In a specific field, a summary document in which content of created documents created daily is summarized after an elapse of a certain period may be created. For example, in the medical field, the summary document is created by summarizing the content of the created documents such as daily medical records at a specific time such as a time of hospital discharge. In a case where a user such as a doctor creates the summary document, providing an ability to present an important examination item and an examination result to the user from the created documents can support creation of the summary document, which is preferable.

A method of outputting an alert in a case where the examination result indicates an abnormal value is known as a method of presenting the important examination item and the examination result. However, the examination result of a patient may indicate an abnormal value for a plurality of examination items. In this case, the examination result is not necessarily important even though the examination result indicates an abnormal value. Thus, the user is required to determine whether or not the examination result is important even in a case where the examination result indicates an abnormal value. In addition, even in a case where the examination result indicates a normal value, the user may be required to refer to content of the examination result or describe the examination result in the summary document. That is, even in a case where the user is notified of the examination result indicating an abnormal value, there is room for improvement from a viewpoint of supporting creation of the summary document.

The present disclosure is conceived in view of the above circumstances, and an object of the present disclosure is to provide a document creation support apparatus, a document creation support method, and a document creation support program that can effectively support creation of a summary document.

A document creation support apparatus according to an aspect of the present disclosure is a document creation support apparatus comprising at least one processor, in which the processor is configured to acquire document information associated with an input target person, derive the number of pieces of examination information included in the document information for each examination item, and derive a degree of importance of the examination item for the target person based on the derived number of pieces of the examination information.

In addition, a document creation support method according to another aspect of the present disclosure is a document creation support method comprising, via a processor included in a document creation support apparatus, acquiring document information associated with an input target person, deriving the number of pieces of examination information included in the document information for each examination item, and deriving a degree of importance of the examination item for the target person based on the derived number of pieces of the examination information.

In addition, a document creation support apparatus according to still another aspect of the present disclosure is a document creation support program for causing a processor included in a document creation support apparatus to execute a process comprising acquiring document information associated with an input target person, deriving the number of pieces of examination information included in the document information for each examination item, and deriving a degree of importance of the examination item for the target person based on the derived number of pieces of the examination information.

According to the present disclosure, creation of a summary document can be effectively supported.

### BRIEF DESCRIPTION OF THE DRAWINGS

FIG. 1 is a block diagram illustrating an example of a configuration of an information processing system.

FIG. 2 is a block diagram illustrating an example of a hardware configuration of a user terminal.

FIG. 3 is a block diagram illustrating an example of a hardware configuration of an information processing apparatus.

FIG. 4 is a diagram illustrating an example of a display screen.

FIG. 5 is a block diagram illustrating an example of a functional configuration of the information processing apparatus in a learning phase.

FIG. 6 is a diagram for describing a prediction model.

FIG. 7 is a diagram for describing a determination model.

FIG. 8 is a flowchart illustrating an example of learning processing.

FIG. 9 is a block diagram illustrating an example of a functional configuration of the information processing apparatus in an operation phase.

FIG. 10 is a flowchart illustrating an example of degree-of-importance derivation processing.

FIG. 11 is a block diagram illustrating an example of a functional configuration of the user terminal.

FIG. 12 is a diagram illustrating an example of a created document display screen.

FIG. 13 is a flowchart illustrating an example of display control processing.

### DETAILED DESCRIPTION

Hereinafter, an embodiment of the disclosed technology will be described in detail with reference to the drawings.

First, a configuration of an information processing system 10 according to the present embodiment will be described with reference to FIG. 1. As illustrated in FIG. 1, the information processing system 10 includes a user terminal 12 used by a user such as a doctor and an information processing apparatus 14. Examples of the user terminal 12 include a tablet computer and a personal computer. Examples of the information processing apparatus 14 include a server computer and a cloud server. The information processing apparatus 14 is an example of a document creation support apparatus according to the disclosed technology.

Each of the user terminal 12 and the information processing apparatus 14 is connected to a network and can transmit and receive data with each other.

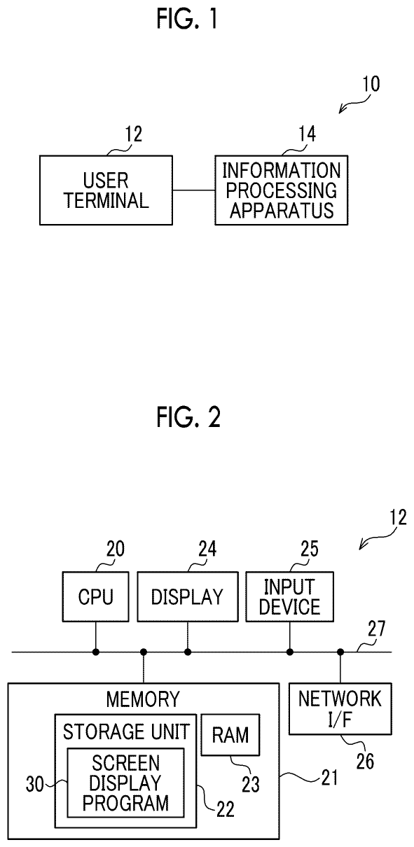

Next, a hardware configuration of the user terminal 12 according to the present embodiment will be described with reference to FIG. 2. As illustrated in FIG. 2, the user terminal 12 includes a central processing unit (CPU) 20, a memory 21, a display 24, an input device 25, and a network interface (I/F) 26.

The CPU 20 implements various functions by executing a program stored in a storage unit 22, described later.

The memory 21 includes the storage unit 22 and a random access memory (RAM) 23. The RAM 23 is a memory for primary storage and is, for example, a RAM such as a static random access memory (SRAM) or a dynamic random access memory (DRAM).

The storage unit 22 is a non-volatile memory and is implemented by, for example, at least one of a hard disk drive (HDD), a solid state drive (SSD), or a flash memory. A screen display program 30 is stored in the storage unit 22 as a storage medium. The CPU 20 reads out the screen display program 30 from the storage unit 22, loads the screen display program 30 into the RAM 23, and executes the loaded screen display program 30.

The screen display program 30 is a program for displaying various screens on the display 24 based on data received from the information processing apparatus 14 through the network I/F 26. The screen display program 30 includes, for example, a web browser program.

The display 24 is a device that displays various screens and is, for example, a liquid crystal display or an electro luminescence (EL) display. The input device 25 is a device for a user to provide input and is, for example, at least any of a keyboard, a mouse, a microphone for audio input, a touchpad for proximity input including a contact, or a camera for gesture input. The network I/F 26 is an interface for connecting to a network. A bus 27 connects the CPU 20, the memory 21, the display 24, the input device 25, and the network I/F 26 to each other.

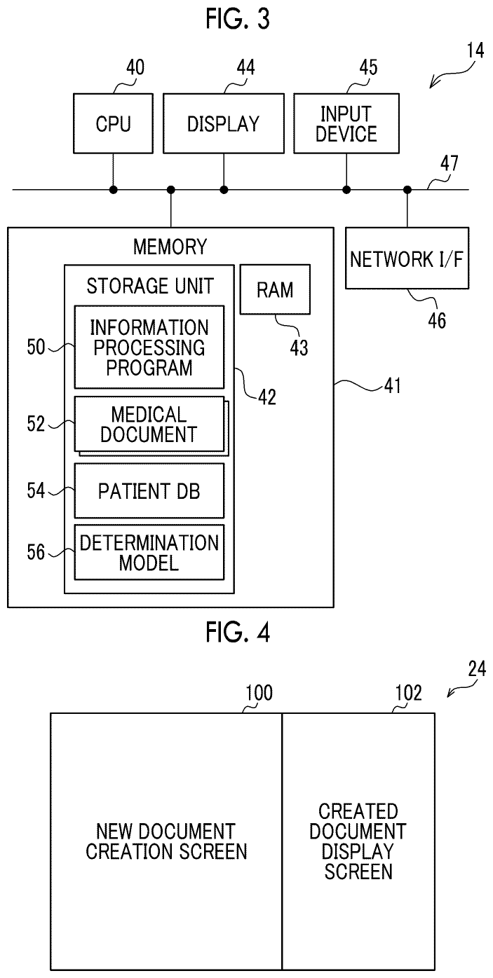

Next, a hardware configuration of the information processing apparatus 14 according to the present embodiment will be described with reference to FIG. 3. As illustrated in FIG. 3, the information processing apparatus 14 includes a CPU 40, a memory 41, a display 44, an input device 45, and a network I/F 46.

The CPU 40 implements various functions by executing a program stored in a storage unit 42, described later. The CPU 40 is an example of a processor according to the disclosed technology.

The memory 41 includes the storage unit 42 and a RAM 43. The RAM 43 is a memory for primary storage and is, for example, a RAM such as an SRAM or a DRAM.

The storage unit 42 is a non-volatile memory and is implemented by, for example, at least one of an HDD, an SSD, or a flash memory. An information processing program 50 is stored in the storage unit 42 as a storage medium. The CPU 40 reads out the information processing program 50 from the storage unit 42, loads the information processing program 50 into the RAM 43, and executes the loaded information processing program 50.

In addition, a plurality of medical documents 52 created by the user are stored in the storage unit 42. The medical document 52 is an example of document information according to the disclosed technology. The plurality of medical documents 52 are used for creating a summary document. In the present embodiment, a case where the user creates the summary document called a hospital discharge summary while viewing the plurality of medical documents 52 will be illustratively described. That is, in the present embodiment, a case where a patient who is discharged from hospital is applied as a target person as a target for creating the summary document will be illustratively described. Examples of the medical document 52 include an electronic medical record. The medical document 52 is learning data in a learning phase, described later, and is actual data in an operation phase.

The medical document 52 is created for each patient, and identification information such as a patient ID of the patient is attached to the medical document 52 as attribute information. Accordingly, which patient is related can be identified. Which patient is related may also be identifiable by inputting the patient ID in a specific input field in the document or storing the medical document 52 in a folder for each patient. Similarly, a creation date of the medical document 52 can also be identified. The medical document 52 is not limited to the electronic medical record and may be a radiology report, a medical fee invoice called a receipt, or the like. In addition, the document information used for creating the summary document is not limited to the medical document and may be, for example, a document such as a research diary. In this case, the user creates a research report by summarizing a plurality of research diaries created daily. In addition, for example, the document information used for creating the summary document may be a daily report, and the summary document may be a weekly report, a monthly report, or the like in which the daily report is summarized.

In addition, a patient database 54 (hereinafter, referred to as a “patient DB 54”) is stored in the storage unit 42. In the patient DB 54, the identification information of the patient and patient information related to the patient are stored in association with each other. Examples of the patient information include information related to the patient such as a sex and an age of the patient, and information related to hospitalization of the patient such as a hospitalization period, a disease name, and an examination result during hospitalization of the patient.

In addition, a determination model 56 is stored in the storage unit 42. The determination model 56 is obtained by performing machine learning in the learning phase, described later. Details of the determination model 56 will be described later.

While a case where the medical document 52, the patient DB 54, and the determination model 56 are stored in the storage unit 42 of the information processing apparatus 14 has been illustratively described in the present embodiment, the disclosed technology is not limited to this aspect. For example, at least one of the medical document 52, the patient DB 54, or the determination model 56 may be stored in a separate data storage server, and at least one of the medical document, the patient information or the like, or a determination result may be acquired through a network.

The display 44 is a device that displays various screens and is, for example, a liquid crystal display or an EL display. The input device 45 is a device for the user to provide input and is, for example, at least any of a keyboard, a mouse, a microphone for audio input, a touchpad for proximity input including a contact, or a camera for gesture input. The network I/F 46 is an interface for connecting to a network. A bus 47 connects the CPU 40, the memory 41, the display 44, the input device 45, and the network I/F 46 to each other.

The user such as a doctor creates the medical document 52 such as the electronic medical record daily with respect to a hospitalized patient and creates a new document called the hospital discharge summary at the specific time such as the time of hospital discharge by summarizing the plurality of medical documents 52 created during a hospitalization period.

As illustrated in FIG. 4, the information processing system 10 according to the present embodiment displays a new document creation screen 100 for creating the new document and a created document display screen 102 for displaying the medical document 52 as an example of a created document to be used for creating the new document, on the display 24 of the user terminal 12.

In a case where the user creates the summary document in which the plurality of medical documents 52 are summarized as a new document, causing the user to search for an important examination item and the examination result from the plurality of medical documents 52 requires time for a search task, and the user cannot efficiently create the summary document. Therefore, the information processing system 10 according to the present embodiment has a function of deriving a degree of importance of an examination item included in the plurality of medical documents 52.

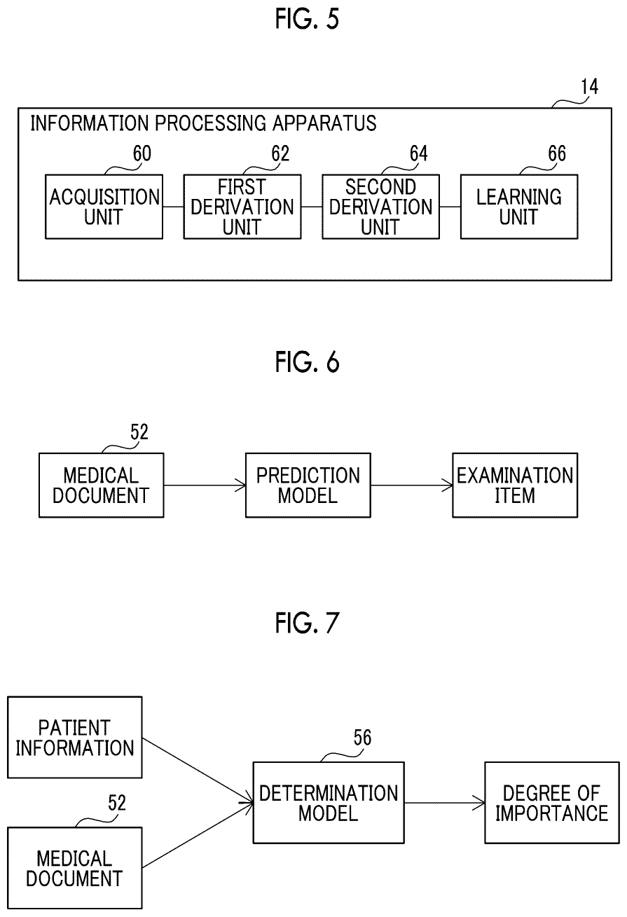

Next, a functional configuration of the information processing apparatus 14 in the learning phase will be described with reference to FIG. 5. As illustrated in FIG. 5, the information processing apparatus 14 includes an acquisition unit 60, a first derivation unit 62, a second derivation unit 64, and a learning unit 66. The CPU 40 functions as the acquisition unit 60, the first derivation unit 62, the second derivation unit 64, and the learning unit 66 by executing the information processing program 50.

The acquisition unit 60 acquires the patient information that is input by the user and that is related to the patient (hereinafter, referred to as a “target patient”) who is the target for creating the new document. Specifically, the acquisition unit 60 acquires the patient information associated with the identification information of the target patient input by the user from the patient DB 54. The patient information is an example of target person information according to the disclosed technology.

In addition, the acquisition unit 60 acquires the medical document 52 associated with the target patient input by the user. Specifically, the acquisition unit 60 acquires, from the storage unit 42, the plurality of medical documents 52 created in a creation target period for the target patient based on the identification information of the target patient and a period (hereinafter, referred to as a “creation target period”) as a target for creating the new document, input by the user. The medical document 52 created in the creation target period is an example of document information created in a specific period according to the disclosed technology. Examples of the creation target period include a hospitalization period in which the patient is hospitalized once. For example, the identification information of the target patient and the creation target period are input into the user terminal 12 by the user and are transmitted to the information processing apparatus 14 from the user terminal 12. The acquisition unit 60 may acquire the hospitalization period of the target patient from the patient DB 54 as the creation target period.

The first derivation unit 62 derives the number of pieces of the examination information included in the plurality of medical documents 52 acquired by the acquisition unit 60 for each examination item. In the present embodiment, a case where the examination information includes a set of an examination date and time, the examination item, and the examination result will be illustratively described. Specifically, the first derivation unit 62 derives the number of pieces of the examination information for each examination item by comparing the examination information with dictionary data in which names of examination items are listed, and counting the number of pieces of the examination information included in the plurality of medical documents 52 for each examination item. In the dictionary data, a plurality of expressions may be associated with one examination item. For example, a plurality of expressions such as “ALB”, “albumin”, “TP”, and “A/G” may be associated with albumin that is an examination item. The dictionary data may be stored in the storage unit 42 or a separate data storage server in advance or may be generated by acquiring an examination item name from input of a new examination item or the like provided by the user or from the examination result included in the patient information. The first derivation unit 62 may compare the examination information with the names of the examination items included in the dictionary data based on a complete match or a partial match. In addition, for example, the first derivation unit 62 may derive the number of pieces of the examination information for each examination item by treating a plurality of examination items of which a degree of similarity is greater than or equal to a certain value as the same examination item. Examples of the degree of similarity in this case include an edit distance, a bilingual evaluation understudy (BLEU) score, a recall-oriented understudy for gisting evaluation (ROUGE) score, or a bidirectional encoder representations from transformers (BERT) score.

More specifically, for example, in a case where the plurality of medical documents 52 include sets of the examination item and the examination result such as “albumin 2.5”, “albumin 2.4”, and “albumin 3.0” as the examination information, the first derivation unit 62 derives 3 as the number of pieces of the examination information corresponding to “albumin” that is the examination item. The first derivation unit 62 may derive the number of pieces of the examination information for each set of the examination item and the examination result. In this case, the first derivation unit 62 derives 1 as the number of pieces of the examination information corresponding to each of “albumin 2.5”, “albumin 2.4”, and “albumin 3.0” in the above example.

As illustrated in FIG. 6, the first derivation unit 62 may derive the number of pieces of the examination information included in the plurality of medical documents 52 for each examination item by inputting the plurality of medical documents 52 acquired by the acquisition unit 60 in an at least partially masked state into a prediction model obtained in advance by machine learning. The prediction model is a model that takes an at least partially masked medical document 52 as input and outputs the examination item included in the input medical document 52. The prediction model is a model obtained by machine learning using a set of the at least partially masked medical document 52 and the examination item included in the medical document 52 collected as learning data. A location to be masked by the first derivation unit 62 in this case may be a part corresponding to the examination item, a part of the examination item, or a randomly selected part. In this case, the number of pieces of the examination information is derived by treating variation in expression of the examination item and an error in the examination item included in the plurality of medical documents 52 as the same examination item.

The second derivation unit 64 derives the degree of importance of the examination item for the target patient based on the number of pieces of the examination information derived by the first derivation unit 62 for each examination item. In the present embodiment, the second derivation unit 64 derives a higher degree of importance of the examination item as the number of pieces of the examination information for each examination item is larger. For example, the second derivation unit 64 derives any of “high”, “medium”, and “low” as the degree of importance of the examination item by setting two different threshold values and comparing the number of pieces of the examination information with the two threshold values. Levels of the degree of importance are not limited to three levels and may be two levels or four or more levels.

The second derivation unit 64 may derive the degree of importance of the examination item for the target patient based on the number of pieces of the examination information derived by the first derivation unit 62 for each examination item and on length information of a sentence including the examination information. The examination information included in the sentence summarized by the user is considered to be relatively important examination information. Therefore, the second derivation unit 64 may derive the degree of importance of the examination item by weighting the number of pieces of the examination information using a weight coefficient having a larger numerical value as a length of the sentence indicated by the length information of the sentence including the examination information is shorter.

In addition, the second derivation unit 64 may derive the degree of importance of the examination item for the target patient based on the number of pieces of the examination information derived by the first derivation unit 62 for each examination item and on the number of types of the examination item included in the plurality of medical documents 52. In a case where the number of types of the examination item included in the plurality of medical documents 52 is small, the examination item that is performed is considered to be a relatively important examination item. Therefore, the second derivation unit 64 may derive the degree of importance of the examination item by weighting the number of pieces of the examination information using the weight coefficient having a larger numerical value as the number of types of the examination item included in the plurality of medical documents 52 is smaller. In addition, an item is considered to be relatively important in a case where the same examination item is derived a plurality of times from different medical documents 52 compared to a case where the same examination item is derived a plurality of times from the same medical document 52. Therefore, the second derivation unit 64 may set the degree of importance to be lower as the number of pieces of the examination information for which the examination item is derived from the same medical document 52 is smaller or the number of medical documents 52 from which the examination item is derived is smaller. For example, the second derivation unit 64 may derive the degree of importance of the examination item by weighting the number of pieces of the examination information using the weight coefficient having a larger numerical value as the number of pieces of the examination information for which the examination item is derived from the same medical document 52 is smaller or the number of medical documents 52 from which the examination item is derived is smaller.

In addition, the second derivation unit 64 may derive the degree of similarity between sentences including the examination information and derive the degree of importance of the examination item for the target patient based on the number of pieces of the examination information derived by the first derivation unit 62 for each examination item and on the derived degree of similarity. For example, in a case where the user creates a new medical document 52, it is considered to copy content of the medical document 52 up to the previous day to the new medical document 52 and then add new content. In this case, since the examination information included in a copied part overlaps with the examination information included in the medical document 52 up to the previous day, the number of pieces of the examination information is relatively increased. Therefore, for the examination information included in a plurality of sentences of which the degree of similarity is greater than or equal to the certain value, the second derivation unit 64 may derive the degree of importance of the examination item by weighting the number of pieces of the examination information using a value less than 1 as the weight coefficient. In addition, for example, in deriving the number of pieces of the examination information, the first derivation unit 62 may determine the plurality of sentences of which the degree of similarity is greater than or equal to the certain value as duplicate sentences and may use one sentence and exclude the rest of the plurality of sentences. In addition, in the medical document 52, an examination item that is periodically performed may be set as a designated description item. In this case, the examination item set as the designated description item is described a large number of times but has a low degree of clinical importance. Thus, the second derivation unit 64 may decrease the degree of importance of the examination item set as the designated description item. For example, the second derivation unit 64 may determine whether or not the derived examination information is the designated description item in the medical document 52 and, in a case where the examination information is determined as being the designated description item, derive the degree of importance of the examination item by weighting the number of pieces of the examination information using the weighting coefficient having a smaller numerical value than that in a case where the examination information is determined as not being the designated description item. The designated description item may be stored as a list designated by the user, or a description item of which the number or a ratio of occurrences among a plurality of patients is greater than or equal to a certain value may be estimated as the designated description item. Since the designated description item may be set in units of any of medical institutions, medical departments, or doctors, a description item of which the number or the ratio of occurrences of patients having any of the medical institution, the medical department, or the doctor in common in the medical document 52 is greater than or equal to the certain value may be estimated as the designated description item.

In addition, each of the medical documents 52 may be associated with a degree of importance of the medical document 52. In this case, the second derivation unit 64 may derive the degree of importance of the examination item for the target patient based on the number of pieces of the examination information derived by the first derivation unit 62 for each examination item and on the degrees of importance associated with the plurality of medical documents 52 acquired by the acquisition unit 60. In this case, the second derivation unit 64 may derive the degree of importance of the examination item by weighting the number of pieces of the examination information using the weight coefficient having a larger numerical value as the degree of importance of the medical document 52 is higher. The degree of importance of the medical document 52 may be output based on a determination model that takes the medical document 52 as input and outputs the degree of importance of the medical document 52, or may be determined using a list or the like in which type information of the medical document 52 such as a nursing record or a medical record associated with the medical document 52 and the degree of importance of the medical document 52 are defined. The list in which the type information and the degree of importance of the medical document 52 are defined may be set in advance or be designated by the user.

In addition, in deriving the degree of importance of the examination item, in a case where numerical value information indicating the examination result of the examination item and text information indicating the examination result of the examination item are related to each other among a plurality of pieces of the examination information including the same examination item, the second derivation unit 64 may weight the number of pieces of the examination information including the text information. In this case, the second derivation unit 64 may perform weighting by multiplying the number of pieces of the examination information including the text information by a numerical value greater than 1 as the weighting coefficient. The examination information indicated by the text information is considered to be important because, for example, an analysis of the examination result provided by the user is included in a case where the examination result included in the examination information is indicated by the text information such as “high CRP value” related to the numerical information compared to a case where the examination result is indicated by the numerical value information such as “CRP 3.0”. Examples of the text information related to the numerical value information of the examination result include a text such as “high” and “low” indicating a magnitude of the numerical value of the examination result, a text such as “increase” and “decrease” indicating a trend of the numerical value of the examination result, and a text such as “negative” and “positive” indicating evaluation of the examination result. The text information described here also includes symbols such as an upward arrow symbol indicating an increase, “+” indicating positivity, and “−” indicating negativity.

In addition, in deriving the degree of importance of the examination item, in a case where the examination result included in the examination information is text information and where the text information indicates strength of the examination result or progress of the examination result, the second derivation unit 64 may weight the number of pieces of the examination information. In this case, the second derivation unit 64 may perform weighting by multiplying the number of pieces of the examination information including the text information indicating the strength of the examination result or the progress of the examination result by a numerical value greater than 1 as the weighting coefficient. The examination information including the text information is considered to be important because, for example, an analysis of the examination result provided by the user is included in a case where a text “strong” indicating the strength of the examination result is added to a text “negativity” indicating the examination result as in “strong negativity”. In addition, the examination information including the text information is considered to be important because, for example, an analysis of the examination result provided by the user is included in a case where a text “continuous” indicating the progress of the examination result is added to the text “negativity” indicating the examination result as in “continuous negativity”.

As illustrated in FIG. 7, the learning unit 66 trains the determination model 56 that takes the patient information and the medical document 52 as input and outputs the degree of importance of the examination item, using the patient information and the medical document 52 acquired by the acquisition unit 60 and the degree of importance of the examination item derived by the second derivation unit 64 for the patient information and the medical document 52 as the learning data. The determination model 56 is, for example, a neural network model and is trained using a well-known algorithm such as backpropagation. The input of the determination model 56 may be any one of the patient information or the medical document 52. In addition, the output of the determination model 56 may include the number of pieces of the examination information used for deriving the degree of importance of the examination item. In addition, the learning data of the learning unit 66 is data in which the examination item that has occurred in the summary document created based on the medical document 52 is set to have a higher degree of importance than the examination item that has occurred in only the medical document 52. For example, the learning unit 66 may use, as the learning data, the degree of importance generated by varying weighting of the degree of importance, which is determined based on the number of occurrences or the ratio of occurrences of the examination item that has occurred in the medical document 52, depending on whether or not the examination item has occurred in the summary document created based on the medical document 52 or on the number of occurrences of the examination item.

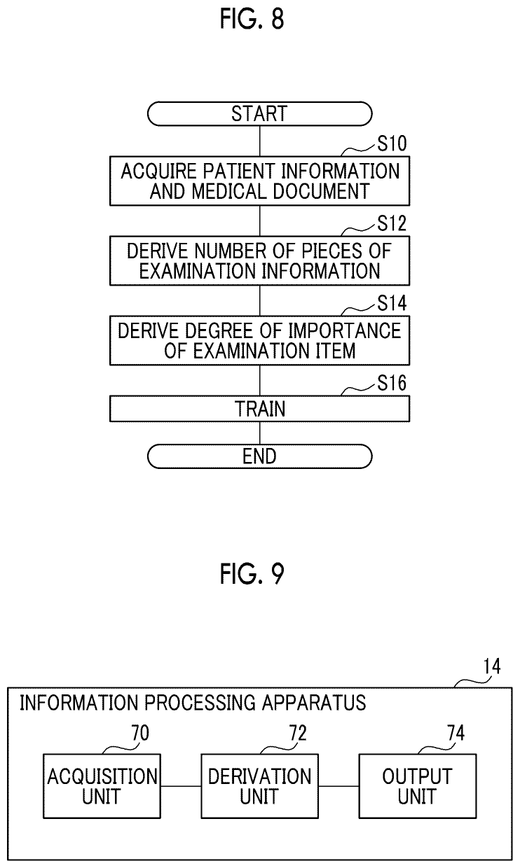

Next, an action of the information processing apparatus 14 in the learning phase will be described with reference to FIG. 8. Learning processing illustrated in FIG. 8 is executed by executing the information processing program 50 via the CPU 40. For example, the learning processing illustrated in FIG. 8 is executed in a case where an execution start instruction is input by the user.

In step S10 in FIG. 8, the acquisition unit 60, as described above, acquires the patient information related to the target patient input by the user. In addition, the acquisition unit 60, as described above, acquires the plurality of medical documents 52 associated with the target patient input by the user. In step S12, the first derivation unit 62, as described above, derives the number of pieces of the examination information included in the plurality of medical documents 52 acquired in step S10 for each examination item.

In step S14, the second derivation unit 64, as described above, derives the degree of importance of the examination item for the target patient based on the number of pieces of the examination information derived in step S12 for each examination item. In step S16, the learning unit 66, as described above, trains the determination model 56 using the patient information and the medical document 52 acquired in step S10 and the degree of importance of the examination item derived for the patient information and the medical document 52 in step S14 as the learning data. In a case where the processing in step S16 is finished, the learning processing is finished. The above learning processing is executed a plurality of times using various types of learning data. In learning of the determination model 56, the same learning data may be used a plurality of times.

Next, a functional configuration of the information processing apparatus 14 in the operation phase will be described with reference to FIG. 9. As illustrated in FIG. 9, the information processing apparatus 14 includes an acquisition unit 70, a derivation unit 72, and an output unit 74. The CPU 40 functions as the acquisition unit 70, the derivation unit 72, and the output unit 74 by executing the information processing program 50.

The acquisition unit 70, like the acquisition unit 60, acquires the patient information related to the target patient input by the user. In addition, the acquisition unit 70, like the acquisition unit 60, acquires the medical document 52 associated with the target patient input by the user.

The derivation unit 72 inputs the patient information and the plurality of medical documents 52 acquired by the acquisition unit 70 into the determination model 56. The determination model 56 outputs the degree of importance of the examination item included in the plurality of input medical documents 52 for the target patient indicated by the input patient information. Accordingly, the derivation unit 72 derives the degree of importance of the examination item included in the plurality of medical documents 52.

The output unit 74 outputs the plurality of medical documents 52 acquired by the acquisition unit 70 and the degree of importance of the examination item derived by the derivation unit 72 to the user terminal 12 through the network I/F 46. The output unit 74 may output only the medical document 52 including the examination information for which the degree of importance of the examination item derived by the derivation unit 72 is greater than or equal to a certain degree, among the plurality of medical documents 52 acquired by the acquisition unit 70 to the user terminal 12. In addition, the output unit 74 may further output the number of pieces of the examination information to the user terminal 12. In this case, the determination model 56 is further trained to output the number of pieces of the examination information.

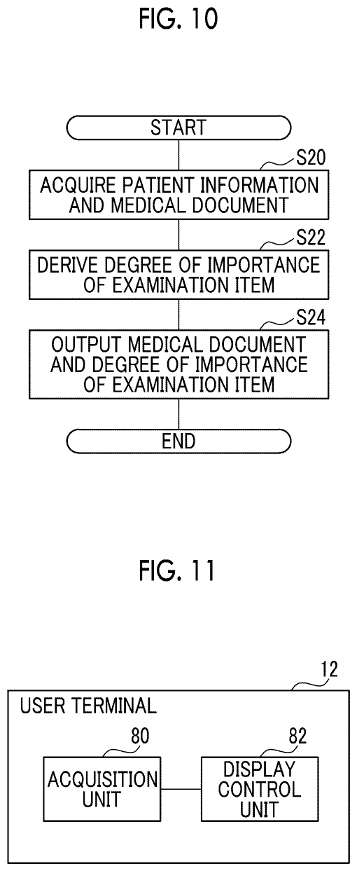

Next, an action of the information processing apparatus 14 in the operation phase will be described with reference to FIG. 10. Degree-of-importance derivation processing illustrated in FIG. 10 is executed by executing the information processing program 50 via the CPU 40. For example, the degree-of-importance derivation processing illustrated in FIG. 10 is executed in a case where an execution start instruction is input by the user.

In step S20 in FIG. 10, the acquisition unit 70, as described above, acquires the patient information related to the target patient input by the user. In addition, the acquisition unit 70, as described above, acquires the plurality of medical documents 52 associated with the target patient input by the user.

In step S22, the derivation unit 72, as described above, derives the degree of importance of the examination item by inputting the patient information and the plurality of medical documents 52 acquired in step S20 into the determination model 56. In step S24, the output unit 74 outputs the plurality of medical documents 52 acquired in step S20 and the degree of importance of the examination item derived in step S22 to the user terminal 12 through the network I/F 46. In a case where the processing in step S24 is finished, the degree-of-importance derivation processing is finished.

Next, a functional configuration of the user terminal 12 will be described with reference to FIG. 11. As illustrated in FIG. 11, the user terminal 12 includes an acquisition unit 80 and a display control unit 82. The CPU 20 functions as the acquisition unit 80 and the display control unit 82 by executing the screen display program 30.

The acquisition unit 80 acquires the plurality of medical documents 52 and the degree of importance of the examination item transmitted from the information processing apparatus 14 through the network I/F 26.

The display control unit 82, as described above, displays the new document creation screen 100 for creating the new document and the created document display screen 102 for displaying the medical document 52 to be used for creating the new document on the display 24 (refer to FIG. 4). In the present embodiment, the display control unit 82 displays at least one piece of the examination information among the pieces of the examination information included in at least any of the patient information or the plurality of medical documents 52, on the created document display screen 102 based on the plurality of medical documents 52 and the degree of importance of the examination item acquired by the acquisition unit 80. Display of the examination information includes, for example, at least one of display of the examination information extracted from at least one of the patient information or the medical document 52 or display of the medical document 52 including the examination information as a display target. In addition, display of the examination information may include display of the medical document 52 in which the examination information as the display target is displayed in a relatively highlighted manner with respect to other pieces of the examination information.

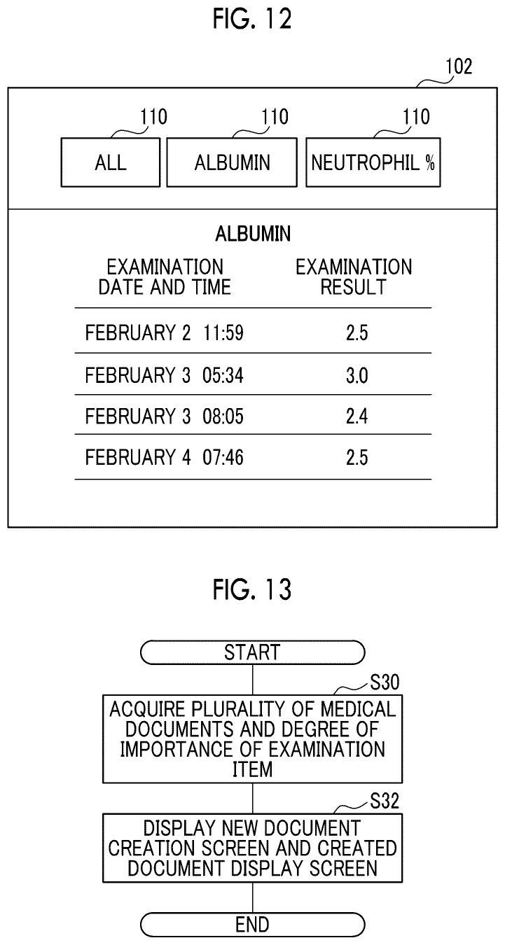

For example, as illustrated in FIG. 12, the display control unit 82 may display the examination information in a summarized state for each examination item on the created document display screen 102. FIG. 12 illustrates an example in which a button 110 for the user to select an examination item and the examination date and time and the examination result corresponding to the examination item selected by the user are displayed on the created document display screen 102. In addition, FIG. 12 illustrates an example in which albumin is selected by the user.

The display control unit 82 may display only the examination information including the examination item of which the degree of importance is greater than or equal to a certain degree among the pieces of the examination information included in the plurality of medical documents 52, on the created document display screen 102. In addition, the display control unit 82 may display the degree of importance of the examination item and the number of pieces of the examination information for each examination item together with the examination information on the created document display screen 102. In addition, the display control unit 82 may vary a display aspect of the examination information in accordance with the degree of importance on the created document display screen 102. Examples of a method of varying the display aspect in this case include a method of varying a background color or a method of varying a display color of a text. For example, the display control unit 82 may display the examination information including the examination item having a relatively high degree of importance in a highlighted manner with respect to the examination information including the examination item having a relatively low degree of importance. For example, in displaying the examination information in a table form, the display control unit 82 may set a background color or a display color of a text of a cell showing the examination information including the examination item having a relatively high degree of importance to be visually noticeable with respect to the examination information including the examination item having a relatively low degree of importance. In addition, in this case, the display control unit 82 may set a background color or a display color of a text of a cell showing the examination information including the examination item having a relatively low degree of importance not to be visually noticeable with respect to the examination information including the examination item having a relatively high degree of importance.

In addition, the display control unit 82 may display the examination information in a descending order or an ascending order of the degree of importance of the examination item on the created document display screen 102. In addition, in a case where the examination information being displayed is selected by the user, the display control unit 82 may display the medical document 52 including the selected examination information on the created document display screen 102. In this case, the display control unit 82 may scroll the medical document 52 so that the examination information including the examination item of which the degree of importance in the medical document 52 is greater than or equal to the certain degree is displayed on the created document display screen 102. In addition, in this case, a mark indicating a position of the examination information including the examination item of which the degree of importance in the medical document 52 is greater than or equal to the certain degree may be displayed on a scroll bar.

In addition, the display control unit 82 may display the plurality of medical documents 52 and display the examination information including the examination item of which the degree of importance in the medical document 52 is relatively high in a highlighted manner with respect to the examination information including the examination item of which the degree of importance is relatively low, on the created document display screen 102. In addition, the display control unit 82 may display only the medical document 52 in which the examination information including the examination item of which the degree of importance is greater than or equal to the certain degree is described among the plurality of medical documents 52, on the created document display screen 102.

In addition, the display control unit 82 may separately display a display region in which the examination information including an important examination result set in advance for each disease is displayed and a display region in which the examination information is displayed based on the degree of importance derived by the information processing apparatus 14, on the created document display screen 102. In addition, the display control unit 82 may specify the examination date that is included in the examination item of which the degree of importance is greater than or equal to the certain degree and that is an examination date of which the number of occurrences in the plurality of medical documents 52 is greater than or equal to a threshold value, on the created document display screen 102. In this case, the display control unit 82 may display the specified examination date in a highlighted manner with respect to other examination dates in displaying a trend graph or the like of the examination result.

Next, an action of the user terminal 12 will be described with reference to FIG. 13. Display control processing illustrated in FIG. 13 is executed by executing the screen display program 30 via the CPU 20. For example, the display control processing illustrated in FIG. 13 is executed in a case where the user terminal 12 receives the plurality of medical documents 52 and the degree of importance of the examination item transmitted from the information processing apparatus 14 through the processing in step S24.

In step S30 in FIG. 13, the acquisition unit 80 acquires the plurality of medical documents 52 and the degree of importance of the examination item transmitted from the information processing apparatus 14 through the network I/F 26. In step S32, the display control unit 82 displays the new document creation screen 100 for creating the new document and the created document display screen 102 for displaying the medical document 52 to be used for creating the new document on the display 24. In this case, the display control unit 82, as described above, displays at least one piece of the examination information among the pieces of the examination information included in the plurality of medical documents 52 based on the plurality of medical documents 52 and the degree of importance of the examination item acquired in step S30, on the created document display screen 102. In a case where the processing in step S32 is finished, the display control processing is finished.

As described above, according to the present embodiment, creation of the summary document can be effectively supported.

While a case where the derivation unit 72 derives the degree of importance of the examination item using the determination model 56 obtained by machine learning has been described in the embodiment, the disclosed technology is not limited to this aspect. For example, the derivation unit 72 may derive the degree of importance of the examination item based on a rule. In this case, the derivation unit 72 may derive the number of pieces of the examination information for each examination item like the first derivation unit 62 and then derive the degree of importance of the examination item based on the number of pieces of the examination information for each examination item like the second derivation unit 64.

In addition, while a case where the first derivation unit 62 derives the number of pieces of the examination information included in the plurality of medical documents 52 for each examination item has been described, the disclosed technology is not limited to this aspect. For example, the first derivation unit 62 may derive the number of pieces of the examination information included in each medical document 52 for each examination item for each medical document 52. In this case, in a case where a common examination item is included in each medical document 52, the first derivation unit 62 may use a representative value of the number of pieces of the examination information including the common examination item derived for each medical document 52 as the number of pieces of the examination information of the examination item. Examples of the representative value in this case include a maximum value, an average value, or a median value.

In addition, in the embodiment, at least a part of functional units comprised in the information processing apparatus 14 may be comprised in the user terminal 12, or at least a part of functional units comprised in the user terminal 12 may be comprised in the information processing apparatus 14. For example, the information processing apparatus 14 may generate the new document creation screen 100 and provide the new document creation screen 100 to the user terminal 12 in a streaming manner. In addition, each functional unit comprised in the user terminal 12 and in the information processing apparatus 14 may be comprised in one computer.

In addition, in the embodiment, the second derivation unit 64 may consider an abnormal value for each examination item when the second derivation unit 64 derives the degree of importance of each examination item. For determining the abnormal values, for example, data for indicating the range of a normal value for each examination item may be stored in the storage unit 42. The second derivation unit 64 may, after extracting the examination result for each examination item, determine whether the examination result is within the range of a normal value. The second derivation unit 64 may derive a higher degree of importance for the examination item corresponding to the examination result determined to be outside the range of a normal value than the examination item corresponding to the examination result determined to be within the range of a normal value. In case that a value of the examination result is outside the range of a normal value, the display control unit 82 may display the examination result and the range of a normal value together with the importance of the examination item so as to make it possible to identify that the examination result is an abnormal value.

In addition, the degree of importance of the examination item derived in the embodiment may be used to support document viewing in addition to being used to support document creation as in the embodiment. In case that the degree of importance is used to support document viewing, the information processing apparatus 14 may extract words of which the degree of importance is greater than or equal to a threshold value, and the user terminal 12 may display the extracted words in a highlighted manner while a user views a document.

In addition, the term “the target person” in this embodiment is not limited to human being. The idea of the term “the target person” includes any creature such as animals and so on.

In addition, in the embodiment, for example, the following various processors can be used as a hardware structure of a processing unit that executes various types of processing, such as each functional unit of the user terminal 12 and each functional unit of the information processing apparatus 14. The various processors include, in addition to a CPU that is a general-purpose processor functioning as various processing units by executing software (program) as described above, a programmable logic device (PLD) such as a field programmable gate array (FPGA) that is a processor having a circuit configuration changeable after manufacture, a dedicated electric circuit such as an application specific integrated circuit (ASIC) that is a processor having a circuit configuration dedicatedly designed to execute specific processing, and the like.

One processing unit may be composed of one of the various processors or may be composed of a combination of two or more processors of the same type or different types (for example, a combination of a plurality of FPGAs or a combination of a CPU and an FPGA). In addition, a plurality of processing units may be composed of one processor.

As an example of the plurality of processing units composed of one processor, first, as represented by a computer such as a client and a server, a form of one processor that is composed of a combination of one or more CPUs and software and that functions as the plurality of processing units is possible. Second, as represented by a system on chip (SoC), a form of using a processor that implements functions of the entire system including the plurality of processing units in one integrated circuit (IC) chip is possible. Accordingly, various processing units are configured using one or more of the various processors as the hardware structure.

Furthermore, more specifically, an electric circuit (circuitry) in which circuit elements such as semiconductor elements are combined can be used as the hardware structure of the various processors.

In addition, while an aspect in which the screen display program 30 is stored (installed) in advance in the storage unit 22 has been described in each embodiment, the disclosed technology is not limited to this aspect. The screen display program 30 may be provided in a form of a recording on a recording medium such as a compact disc read only memory (CD-ROM), a digital versatile disc read only memory (DVD-ROM), and a universal serial bus (USB) memory. In addition, the screen display program 30 may be provided in a form of a download from an external apparatus through a network.

In addition, while an aspect in which the information processing program 50 is stored (installed) in advance in the storage unit 42 has been described in the embodiment, the disclosed technology is not limited to this aspect. The information processing program 50 may be provided in a form of a recording on a recording medium such as a CD-ROM, a DVD-ROM, and a USB memory. In addition, the information processing program 50 may be provided in a form of a download from an external apparatus through a network.

The following appendixes are further disclosed with respect to the above embodiment.

### Appendix 1

A document creation support apparatus comprising at least one processor, in which the processor is configured to acquire document information associated with an input target person, derive the number of pieces of examination information included in the document information for each examination item, and derive a degree of importance of the examination item for the target person based on the derived number of pieces of the examination information.

### Appendix 2

The document creation support apparatus according to Appendix 1, in which the processor is configured to further acquire target person information related to the target person, and derive the number of pieces of the examination information included in the document information created in a specific period for each examination item based on the target person information.

### Appendix 3

The document creation support apparatus according to Appendix 2, in which the processor is configured to train a determination model that takes at least one of the target person information or the document information as input and outputs the degree of importance of the examination item, using at least one of the acquired document information or the acquired target person information and a degree of importance derived for the at least one of the document information or the target person information as learning data.

### Appendix 4

The document creation support apparatus according to any one of Appendixes 1 to 3, in which the processor is configured to derive the number of pieces of the examination information included in the acquired document information by inputting the acquired document information in an at least partially masked state into a prediction model that takes at least partially masked document information as input and outputs an examination item and that is obtained in advance by machine learning.

### Appendix 5

The document creation support apparatus according to any one of Appendixes 1 to 4, in which the processor is configured to derive the degree of importance based on the derived number of pieces of the examination information and on length information of a sentence including the examination information.

### Appendix 6

The document creation support apparatus according to any one of Appendixes 1 to 5, in which the processor is configured to derive the degree of importance based on the derived number of pieces of the examination information and on the number of types of the examination item included in the document information.

### Appendix 7

The document creation support apparatus according to any one of Appendixes 1 to 6, in which the processor is configured to derive a degree of similarity between sentences including the examination information, and derive the degree of importance based on the derived number of pieces of the examination information and on the derived degree of similarity.

### Appendix 8

The document creation support apparatus according to any one of Appendixes 1 to 7, in which the document information is associated with a degree of importance of the document information, and the processor is configured to derive the degree of importance of the examination item for the target person based on the derived number of pieces of the examination information and on a degree of importance associated with the acquired document information.

### Appendix 9

The document creation support apparatus according to any one of Appendixes 1 to 8, in which the processor is configured to, in a case where numerical value information indicating an examination result of the examination item and text information indicating an examination result of the examination item are related to each other among a plurality of pieces of the examination information including the same examination item, weight the number of pieces of the examination information including the text information.

### Appendix 10

The document creation support apparatus according to any one of Appendixes 1 to 8, in which the processor is configured to, in a case where an examination result included in the examination information is text information and where the text information indicates strength of the examination result or progress of the examination result, weight the number of pieces of the examination information.

### Appendix 11

A document creation support method comprising, via a processor comprised in a document creation support apparatus, acquiring document information associated with an input target person, deriving the number of pieces of examination information included in the document information for each examination item, and deriving a degree of importance of the examination item for the target person based on the derived number of pieces of the examination information.

### Appendix 12

A document creation support program for causing a processor comprised in a document creation support apparatus to execute a process comprising acquiring document information associated with an input target person, deriving the number of pieces of examination information included in the document information for each examination item, and deriving a degree of importance of the examination item for the target person based on the derived number of pieces of the examination information.

What is claimed is:

1. A support apparatus comprising:
at least one processor,
wherein the processor is configured to:
acquire document information associated with an input target person;
derive the number of pieces of examination information included in the document information for each examination item; and
derive a degree of importance of the examination item for the target person based on the derived number of pieces of the examination information.

2. The support apparatus according to claim 1,
wherein the processor is configured to:
further acquire target person information related to the target person; and
derive the number of pieces of the examination information included in the document information created in a specific period for each examination item based on the target person information.

3. The support apparatus according to claim 2,
wherein the processor is configured to train a determination model that takes at least one of the target person information or the document information as input and outputs the degree of importance of the examination item, using at least one of the acquired document information or the acquired target person information and a degree of importance derived for the at least one of the document information or the target person information as learning data.

4. The support apparatus according to claim 1,
wherein the processor is configured to derive the number of pieces of the examination information included in the acquired document information based on an examination item output from a prediction model by inputting the acquired document information in an at least partially masked state into the prediction model; wherein the prediction model is trained in advance by machine learning so as to take at least partially masked document information as input and output the examination item.

5. The support apparatus according to claim 1,
wherein the processor is configured to derive the degree of importance based on the derived number of pieces of the examination information and on length information of a sentence including the examination information.

6. The support apparatus according to claim 1,
wherein the processor is configured to derive the degree of importance based on the derived number of pieces of the examination information and on the number of types of the examination item included in the document information.

7. The support apparatus according to claim 1,
wherein the processor is configured to:
derive a degree of similarity between sentences including the examination information; and
derive the degree of importance based on the derived number of pieces of the examination information and on the derived degree of similarity.

8. The support apparatus according to claim 1,
wherein the document information is associated with a degree of importance of the document information, and
the processor is configured to derive the degree of importance of the examination item for the target person based on the derived number of pieces of the examination information and on a degree of importance associated with the acquired document information.

9. The support apparatus according to claim 1,
wherein the processor is configured to derive the degree of importance based on the derived number of pieces of the examination information and a degree of association between numerical value information indicating an examination result of the examination item and text information indicating an examination result of the examination item.

10. The support apparatus according to claim 1,
wherein the processor is configured to derive the degree of importance based on the derived number of pieces of the examination information and a result of determination of whether an examination result included in the examination information is text information indicating strength of the examination result or progress of the examination result.

11. A support method comprising:
via a processor included in a support apparatus,
acquiring document information associated with an input target person;
deriving the number of pieces of examination information included in the document information for each examination item; and
deriving a degree of importance of the examination item for the target person based on the derived number of pieces of the examination information.

12. A non-transitory computer-readable storage medium storing a support program for causing a processor included in a support apparatus to execute a process comprising:
acquiring document information associated with an input target person;
deriving the number of pieces of examination information included in the document information for each examination item; and
deriving a degree of importance of the examination item for the target person based on the derived number of pieces of the examination information.
